(19) United States
(12) Patent Application Publication
(10) Pub. No.: US 2003/0092657 A1
(43) Pub. Date: May 15, 2003

(54) Modulation of PAX-2 for controlled apoptosis or survival of cells

(76) Inventors: Paul Goodyer, Montreal West (CA); Roger Michael Eccles, Mosgid (NZ); Elena Torban, Brossard (CA)

Correspondence Address: David A. Jackson, KLAUBER & JACKSON, 4th Floor, 411 Hackensack Street, Hackensack, NJ, 07601, US

(21) Appl. No.: 10/175,138

(22) Filed: Jun. 19, 2002

Related U.S. Application Data
(63) Continuation of application No. PCT/CA00/01545, filed on Dec. 21, 2000.
(60) Provisional application No. 60/171,443, filed on Dec. 22, 1999.
(60) Provisional application No. 60/220,161, filed on Jul. 24, 2000.

(51) Int. Cl. A61K 48/00; A61K 39/395

(52) U.S. Cl. 514/44; 424/146.1

(57) ABSTRACT

The present invention relates to a method of modulating resistance to apoptosis in a patient. The treatment consists of administering to a patient a therapeutically effective amount of an enhancing agent which selectively inhibits or stimulates the function of PAX-2. The enhancing agent of the present invention allows to increase or to reduce resistance to apoptosis depending of targeted cells.

### BACKGROUND OF THE INVENTION

(a) Field of the Invention

The invention relates to modulators of PAX-2 gene for controlled therapeutical apoptosis of cells or therapeutical survival of cells.

(b) Description of Prior Art

PAX2 is a transcription factor critically required during the development of the nervous and excretory systems, including the mid/hindbrain, spinal cord, eye, ear and urogenital tract (Dressler, G. R. et al. (1990) Development, 109, 787-795; Eccles, M. R. et al. (1992) Cell Growh & Diff., 3, 279-289; Dahl, E. et al. (1997) BioEssays, 19, 755-764). Like other members of the PAX gene family, PAX2 encodes a conserved 128 amino acid paired box DNA binding domain in the N-terminal portion of the molecule (Dahl, E. et al. (1997) BioEssays, 19, 755-764). While PAX2 is a transcriptional regulator, there are no proven target genes regulated by PAX2, and little is yet known of the exact role of PAX2 during development of the nervous or excretory systems.

Eight of the nine Pax genes cause phenotypic abnormalities when mutated in humans or mice, and in four of these (including abnormalities caused by PAX2 mutations) developmental abnormalities are observed in the heterozygotes, revealing haploinsufficiency (Dahl, E. et al. (1997) BioEssays, 19, 755-764; Sanyanusin, P. et al. (1995) Nature Genet., 9, 358-364). To date all PAX2 mutations in humans have occurred within the conserved paired box and octapeptide sequences contained in the 5′ half of the gene (refer to the human PAX2 sequence variant database online) (Sanyanusin, P. et al. (1995) Nature Genet., 9, 358-364). The 3′ half of the PAX2 gene, which covers the remaining 7 of the 12 exons including alternative splices in exons 6, 10 and 12, encodes a putative transactivation domain.

In previous reports, patients with PAX2 mutations have been noted to have optic nerve colobomas and renal hypoplasia (renal-coloboma syndrome) (Sanyanusin, P. et al. (1995) Nature Genet., 9, 358-364). This syndrome has mostly been characterized within the last 10 years, and is associated with number of less common features, including high frequency hearing loss, seizure and brain malformation disorders, joint and skin anomalies and vesico-uretleral Ad reflux (VUR) (Sanyanusin, P. et al. (1995) Nature Genet., 9, 358-364). The renal phenotype associated with renal-coloboma syndrome is frequently accompanied by end-stage renal failure, often necessitating renal transplant. While it is clear that PAX2 plays a critical role during kidney development, the exact role is unknown, and our present understanding of the pathogenesis of renal failure as a result of PAX2 mutations is poor.

During embryonic life the ureteric bud emerges from the nephric duct, growing outward into and arborizing within the undifferentiated mesenchyme. Signals from each branch of the ureteric bud induce adjacent mesenchymal cells to transform into proximal tubules and glomeruli of individual nephrons which ultraflilter fluid from blood. Each nephron fuses with its parent branch of the ureteric bud, providing an outlet to the bladder. Thus the size and functional capacity of each kidney is ultimately determined by the complexity of ureteric bud branching and the number of individual nephrons which have been induced by the time the pool of metanephric stem cells has been consumed and nephrogenesis is terminated.

During normal kidney development PAX2 is expressed throughout the branching ureteric bud, Wolffian and Mullerian ducts. PAX2 is subsequently expressed in each focus of induced nephrogenic mesenchyme and its derivatives, the early epithelial structures of the emerging nephron (Dressler, G. R. et al. (1990) Development, 109, 787-795; Eccles, M. R. et al. (1992) Cell Growth & Diff., 3, 279-289). The importance of PAX2 to nephrogenesis is evident in mice with targeted disruption or spontaneous mutations of the Pax2 gene (Dahl, E. et al. (1997) BioEssays, 19, 755-764; Eccles, M. R. (1998) Pediatr. Nephrol., 12, 712-720). Heterozygous mutants have a phenotype very similar to the human PAX2 mutation syndrome (renal-coloboma syndrome) and are able to reproduce. Homozygous Pax2 mutant mice lack kidneys, ureters, vas deferens, epidydimis, seminal vesicles, uterus, oviducts and vagina and have developmental defects of the eyes, ears and CNS; these defects are lethal in the perinatal period (Eccles, M. R. (1998) Pediatr. Nephrol., 12, 712-720).

To gain insight into the cause of renal abnormalities in patients with PAX2 mutations and to understand how these abnormalities lead to renal failure, we initially focused on the renal phenotype in series of patients with renal-coloboma syndrome. A new PAX2 mutation was identified in this study, which was a novel stop codon mutation in PAX2 exon 7 in 9 members of a large Brazilian pedigree spanning three generations. We also identified the common G619 insertion mutation in exon 2 of PAX2 in a sporadic Japanese patient. The renal phenotypes in a total of 29 renal-coloboma syndrome patients were then compared, identifying renal hypoplasia as the most common congenital renal abnormality. To characterize the etiology of the renal hypoplasia, fetal kidneys of mice carrying a Pax2^{1Neu} mutation (Koseki, C. et al. (1992) J. Cell Biol., 119, 1327-1333) were analyzed. This mutation is identical to the G619 insertion mutation in some humans. The fetal kidney size of heterozygous mutants was reduced to 60% of that of wild-type littermates, closely resemblinc renal hypoplasia in humans. Heterozygous 1Neu mice showed reduced branching and increased apoptotic cell death in fetal kidney collecting ducts, but the increased apoptosis was not associated with random stochastic inactivation of Pax2 expression in the mutant kidneys, and Pax2 was biallelically expressed during kidney development. The extent of the apoptosis correlated well temporally and spatially with the known pattern of PAX2 expression, tapering off when PAX2 expression levels are known to decline. Our findings support the notion that heterozygous PAX2 mutations lead to increased apoptosis and reduced branching of the ureteric bud during a critical window in kidney development.

It would be highly desirable to be provided with modulators of PAX-2 gene for controlled therapeutical apoptosis of cells or therapeutical survival of cells.

### SUMMARY OF THE INVENTION

One aim of the present invention is to provide modulators of PAX-2 gene for controlled therapeutical apoptosis of cells or therapeutical survival of cells.

In accordance with the present invention there is provided a method of treating cancer and cystic kidney disease in a patient comprising the step of administering to said patient a therapeutically effective amount of an agent which selectively prevents the function of PAX-2.

The agent prevents the expression of PAX-2, such as an antisense of PAX-2.

The agent may inhibit the activity of PAX-2, such as a ribozyme.

Cancer treated is selected from the group consisting of prostate, ovarian, bladder and kidney cancers.

In accordance with the present invention there is provided a method of rescuing cells from apotosis in a patient comprising the step of administering to said patient a therapeutically effective amount of an agent which selectively stimulate the function of PAX-2.

The agent may activate the expression of PAY-2.

In accordance with the present invention there is provided a method to enhance resistance of normal tissues to apoptotic cell death induced by chemotherapy or radiation therapy in a patient comprising the step of administering to said patient a therapeutically effective amount of an agent which selectively stimulate the function of PAX-2.

The agent may activate the expression of PAY-2.

In accordance with the present invention there is provided a genetic construct comprising a nucleic acid encoding a molecule capable of preventing or stimulating the function of PAX-2.

In accordance with the present invention there is provided a method of diagnosing prostate, ovarian, bladder and/or kidney cancer, renal hypoplasia, renal failure, renal-coloboma and cystic kidney diseases in a patient comprising the steps of

a) obtaining a sample containing nucleic acid or protein from prostate, ovary, bladder and/or kidney cells or tissue of said patient; and

b) determining whether the sample contains a PAX-2 mutation or a level of PAX-2 nucleic acid or protein associated with inappropriate postnatal PAX-2 overexpression, thereby being indicative of neoplast c phenotype or disease phenotype associated with abnormal PAX-2 function.

### BRIEF DESCRIPTION OF THE DRAWINGS

FIG. 1 illustrates the pedigree of the Brazilian family with renal-coloboma syndrome;

FIG. 2 illustrates the detection of PAX2 mutations in the Brazilian family, and in a sporadic Japanese patient;

FIG. 3 illustrates a graph of kidney size in 10 patients with PAX2 mutations;

FIG. 4 illustrates the morphology of E15 1Neu mutant and wild type kidneys;

FIG. 5 illustrates the analysis of nephrogenesis in E15 1Neu mutant and wild type kidneys;

FIG. 6. illustrates the analysis of apoptosis and cell proliferation in fetal kidneys from Pax2^{1Neu}/+ heterozygous mutant and wild type mice;

FIG. 7 illustrates graphs showing the number of nick-end-labeled cells per kidney area in fetal and postnatal kidneys from Pax21Neu/+ mutant and wildtype mice; and

FIG. 8 illustrates biallelic expression of Pax2 in fetal kidney epithelial cells;

### DETAILED DESCRIPTION OF THE INVENTION

PAX2 mutations cause renal-coloboma syndrome (RCS), a rare multi-system developmental abnormality involving optic nerve colobomas and renal abnormalities. End-stage renal failure is common in RCS, but the mechanism of how PAX2 mutations lead to renal failure is unknown. PAX2 is a member of a family of developmental genes containing a highly conserved “paired box” DNA-binding domain, and encodes a transcription factor expressed primarily during Fetal development in the CNS, eye, ear and urogenital tract. Presently, the role of PAX2 during kidney development is poorly understood. To gain insight into the cause of renal abnormalities in patients with PAX2 mutations, kidney anomalies were analyzed in patients with RCS, including a large Brazilian kindred in whom a new PAX2 mutation was identified. In a total of 29 patients renal hypoplasia was the most common congenital renal abnormality. To determine the direct effects of PAX2 mutations on kidney development fetal kidneys of mice carrying a Pax2^{1Neu} mutation were examined. At E15, heterozygous mutant kidneys were about 60% of the size of wild-type littermates, and the number of nephrons was strikingly reduced. Heterozygous 1Neu mice showed increased apoptotic cell death during fetal kidney development, but the increased apoptosis was not associated with random stochastic inactivation of Pax2 expression in mutant kidneys; Pax2 was shown to be biallelically expressed during kidney development. These findings support the notion that heterozygous mutations of PAX2 are associated with increased apoptosis and reduced branching of the ureteric bud, due to reduced PAX2 dosage during a critical window in kidney development.

### Materials and Methods

### PCR-SSCP and Detection of PAX2 Mutations

Genomic DNA from each individual was extracted from peripheral blood (collected with informed consent) using a DNA extraction kit (Promega). Fragments spanning exons 1-12 of PAX2 were amplified from genomic DNA using PCR primers in the introns flanking the exons. The PCR products were labeled by incorporation of ³²P-dCTP in reactions containing 100 ng DNA, 62.5 μM dNTPs, 1 μCi [α-³²P] dCTP (3000 Ci/mmol), 20 pmol of each primer, reaction buffer (50 mM KCl, 10 mM Tris pH 8.3, 1.0-3.0 mM MgCl₂) and Pwo (Boehringer) or AmpliTaq Gold (Perkin Elmer) DNA polymerase, and electrophoresed in non-denaturing 6% or 12% polyacrylamide gels as described previously (Sanyanusin, P. et al. (1995) Nature Genet., 9, 358-364) to reveal single-strand conformational variants.

### Subcloning and DNA Sequencing

PCR products were directly sequenced using γ³³P-ATP radiolabeled kinased primers and a cycle sequencing kit (GIBCO-BRL). For subcloning of exon 2 and exon 7 PCR products, mutant and normal alleles of PAX2 were amplified using Pwo DNA polymerase (Boehringer Mannheim), and subcloned into EcoRV digested pBluescript II (Stratagene). Positive clones were identified by blue/white selection, and plasmid DNA was isolated using Wizard mini-preps kits (Promega). Sequencing reactions were carried out on plasmid DNA as described previously (Sanyanusin, P. et al. (1995) Nature Genet., 9, 358-364).

### Mouse Breeding, Genotyping and Embryo Preparation

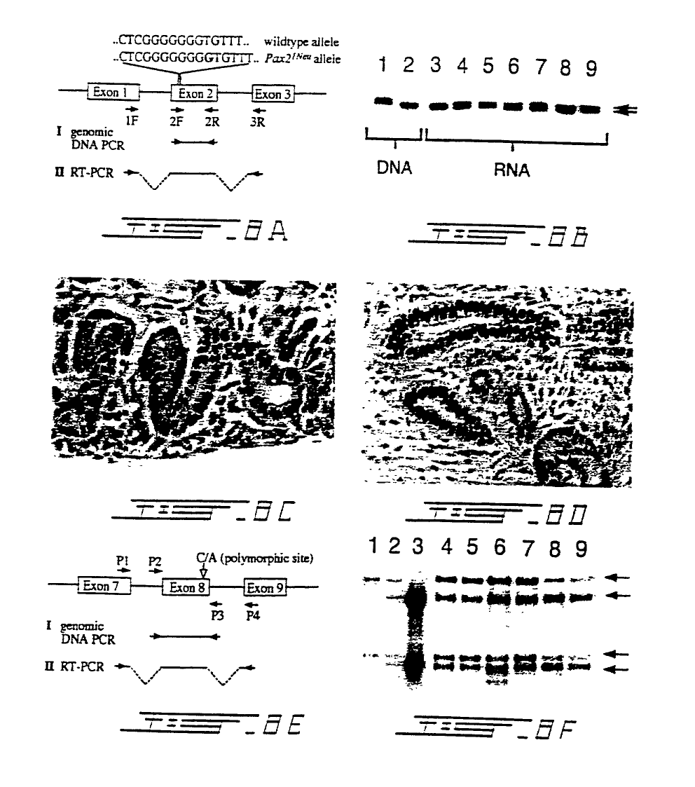

Wild type and 1Neu mouse colonies were bred and maintained at Neuherberg, Germany and at University of Otago, New Zealand. Timed matings of male mice heterozygous for the Pax2^{1Neu} mutant allele (Koseki, C. et al. (1992) J. Cell Biol., 119, 1327-1333) were carried out with female C57BL/6 mice, and the embryos were genotyped. Genotyping was performed as described (Koseki, C. et al. (1992) J. Cell Biol., 119, 1327-1333), or by PCR amplification of genomic DNA extracted from tail slices by using primers 2F and 2R (see FIG. 8). PCR amplification reactions were as previously described (Sanyanusin, P. et al. (1995) Nature Genet., 9, 358-364). The presence of a 167 bp fragment in addition to a -66 bp fragment when the PCR products were electrophoresed on a 6% polyacrylamide gel indicated the presence of the mutant Pax2 allele. Fetal kidneys (E15-E18) were dissected from heterozygous Pax2^{1Neu}/+ mutant or wild-type mice. The expression of the mutant Pax2^{1Neu} allele was demonstrated in fetal kidney RNA from Pax2^{1Neu}/+ mutant mice by RT-PCR amplification of total kidney RNA using primers 1F and 3R, spanning exons 1-3 of Pax2. Nested PCR amplification of the RT-PCR product from exons 1-3 was carried out using primers 2F and 2R. PCR products were electrophoresed on 6% polycrylamide gels. Primer sequences were based on the murine Pax2 sequence (Dressler, G. R. et al. (1990) Development, 109, 787-795) and were as follows;

1F, 5′-CCA CCG TCC CTC CCT TTT CTC CT-3′;

2F, 5′GGG CAC GGG GGT GTG AAC CAG-3′;

2R, 5′-CTG CCC AGG ATT TTG CTG ACA CAG CC-3′;

3R, 5′-CTG TGT CAT TGT CAC A-A TGC CCT CG-3′.

### Microscopic Analysis

Heterozygous PAX2 mutant E15 embryos and their wild type littermates were incubated in freshly prepared 4% paraformaldehyde in PBS for 16-18 hours and stored at 4° in 70% ethanol prior to embedding in paraffin. Serial 5 μm sagital sections of both kidneys from each mutant and wild type embryo were prepared on Superfrost/Plus slides (Fisher). Sections were stained with hematoxylin-eosin and analyzed by bright field microscopy: the number of glomeruli and early epithelial structures (renal condensates, comma- and S-shaped bodies) in the nephrogenic layer was counted in serial cross-sections from both kidneys of 1Neu and wild type embryos. To compare the maximal cross-sectional area of heterozygous and wild type kidneys, serial cross-sections were visualized by photomicroscopy and projected on a television screen. The outline of each renal image was traced onto paper, cut out and weighed to estimate relative surface area. The maximal cross-sectional area for each kidney was plotted in arbitrary units for inter-kidney comparisons.

### PAX2 Immunostaining

Polyclonal anti-mouse PAX2 antibody was purchased from Zymed, California. Mouse sections were dezaraffinized and incubated with primary antibody (10 μg/ml) for 30 min. at room temperature in PBS supplemented with 20 mM glycine and 1% BSA. PAX2 antibody was detected with Vecstatin ABC Rabbit IaG kit (Vector Laboratories) as described by the manufacturer, followed by incubation with DAB substrate (Sigma). Sections were washed in PBS, counterstained with hematoxylin, dehydrated and mounted with Permount (Fisher). Wild type and mutant tissues were stained simultaneously; each analysis was repeated 3 times.

### Detection of Apoptosis (TUNEL Staining)

The TUNEL assay was performed on paraffin-embedded tissue sections as described (Gavrielli, Y. et al. (1992) J. Cell Biol., 119, 493-501) with modifications. The paraffin sections were dewaxed, rehydrated, partially digested with proteinase K (15 μg/ml; Boehringer Mannheim) in Tris·Cl (10 mM, pH 7.6) at room temperature (RT) for 15 minutes. After the endogenous peroxidase was inactivated with 2% H₂O₂ for 10 minutes at RT, the sections were incubated for 90 minutes at 37° C. with terminal deoxynucleotidyl transferase (TdT, Boehringer Mannheim) in TdT reaction buffer (1 mM dCTP, 1 mM dGTP, 1 mM TTP, 1 mM biotin-14-dATP, 140 mM sodium cacodylate, 1 mM cobalt chloride, 30 mM Tris-HCl, pH 6.4). The sections were then washed 3 times in PBS, incubated with extravidin peroxidase (1:250 dilution in PBS, SIGMA) for 60 minutes at RT, rinsed 3 times in PBS, stained with diaminobenzidine (Boehringer Mannheim) for 5 minutes at RT, and counterstained with 1% methyl green.

### BrdU Incorporation Studies and PCNA Immunostaining

BrdU incorporation studies were performed exactly as described (Gavrielli, Y. et al. (1992) J. Cell Biol., 119, 493-501). For PCNA immunostaining mouse sections were deparaffinized, hydrated, and endogenous neroxidase activity was quenched by incubating in methanol containing 0.3% H₂O₂. Slides were incubated in 2.5 μg/ml PCNA monoclonal antibody (Oncoaene Research) at room temperature for 30 min followed by two brief washes in PBS. PCNA antibody was detected with Vecstatin Mouse IgG kit (Vector Laboratories) as described by the manufacturer followed by incubation with DAB substrate (Sigma).

### Results

### Renal Abnormalities in 29 Patients with PAX2 Mutations

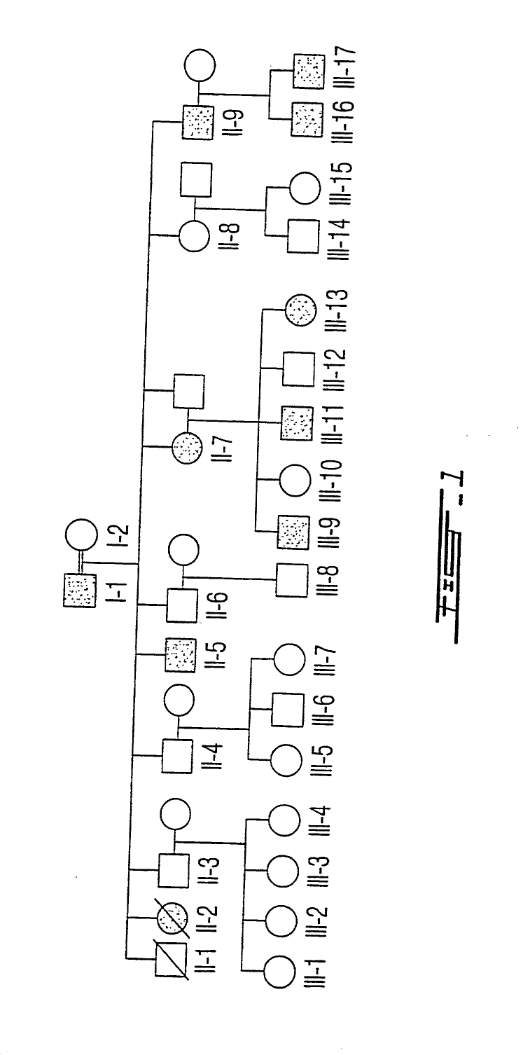

To further characterize the renal phenotype associated with renal-coloboma syndrome, renal features were compared in a total of 29 patients with PAX2 mutations, including 10 new patients from 2 families. The 10 new patients are described in the following. A three generation Brazilian kindred (pedigree shown in FIG. 1) was evaluated for optic nerve and kidneys defects consistent with diagnosis of renal-coloboma syndrome. Individuals in three generations of the pedigree (I, II and III) are shown in FIG. 1. Filled symbols represent affected individuals. Open symbols represent unaffected individuals.

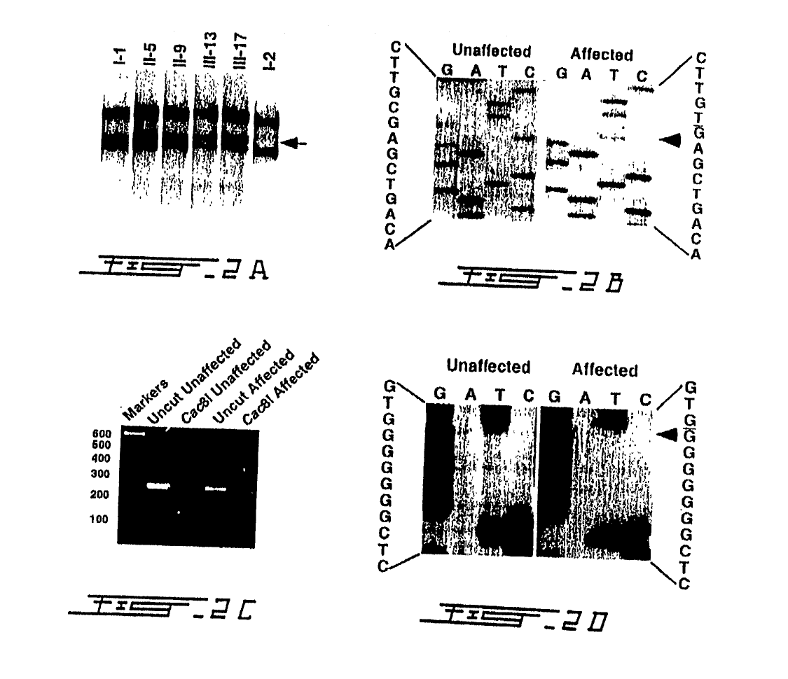

The associated phenotypic abnormalities are listed in Table 1. PCR-SSCP revealed a variant pattern in exon 7 that was present in 5 of the affected members of the family, but was not Present in 18 unaffected members (FIG. 2A). PCR-SSCP analysis of PAX2 exon 7 showed the SSCP variant band in the affected individuals, but not in the unaffected individual (arrow; FIG. 2A).

Independently derived mutant alleles from the exon 7 PCR product contained a cytosine to thymine substitution at position 1289 (FIG. 2B). The mutant and normal PAX2 sequence were Identified in exon 7 of affected members of the Brazilian family. The mutation (arrowhead) is a C→T substitution at position 1289 (underlined) of the PAX2 cDNA sequence (Eccles, H. R. et al. (1992) Cell Growth & Diff., 3, 279-289) (FIG. 2B).

Normal alleles were also identified in DNA from these individuals, while unaffected patients did not contain the mutation. In exon 7 the nucleotide substitution at this position resulted in the disruption of a Cac8I restriction endonuclease site due to the change from a GCNNGC recognition motif to GTNNGC. Restriction digestion with Cac8I of exon 7 PCR products from one of the affected Brazilian family members showed sequences resistant to digestion as well as sequences which could be digested by Cac81. FIG. 2C shows the Cac81 restriction enzyme digestion of the PCR products from affected and unaffected members of the Brazilian family. The undigested band in the uncut unaffected, and affected lanes and in the Cac81 digested affected lanes is 234 bp, which is digested to products of 137 and 97 bp by Cac81.

Unaffected family members contained only sequences that digested with Cac81. Additionally, a Japanese patient was examined who had sporadic occurrence of optic nerve colobomas, renal anomalies and bilateral cryptorchidism (see Table 1, patient X2003 for phenotypic details).

TABLE 1
Summary of phenotypic abnormalities in 9 members of the Brazilian
family

| Patient | Age | Sex | Phenotype |
|---|---|---|---|
| I-1 | 70 | M | Abdominal US normal, systemic hypertension, serum creatinine |
| | | | 1.8 mg/dL, blood urea 116 mg/dL, left mild sensorineural hearing |
| | | | loss, optic pit right eye, optic nerve coloboma left eye, left eye |
| | | | visual acuity 20/20, right eye counting fingers (CF). |
| II-2 | deceased | F | History of a probable renal disorder |
| II-5 | 37 | M | Abdominal US showed bilateral small kidneys, and cortical |
| | | | hyperechogenicity, renal failure over past 2 years, bilateral optic |
| | | | nerve colobomas, visual acuity was CF in OD and 20/20 in OS. |
| II-7 | 40 | F | No renal dysfunction, abdominal US normal, bilateral optic nerve |
| | | | colobomas, visual acuity was 20/20 in both eyes. |
| II-9 | 39 | M | History of bilateral nephrolithiasis, previous episode of obstructive |
| | | | urolithiasis in left ureter, single functioning right kidney, serum |
| | | | creatinine 1.7 mg/dL, blood urea 54 mg/dL, bilateral optic nerve |
| | | | colobomas, visual acuity was 20/20 in OD and CF in OS. |
| III-9 | 14 | M | No renal dysfunction, abdominal US normal, bilateral optic |
| | | | papillae dysplasia, visual acuity 20/20 in both eyes. |
| III-11 | 18 | M | No renal dysfunction, abdominal US normal, bilateral optic nerve |
| | | | colobomas, visual acuity 20/20 in both eyes. |
| III-13 | 21 | F | Abdominal US showed bilateral small kidneys with cystic |
| | | | appearance in upper pole, hyperechogenicity of renal cortex and |
| | | | no medullary differentiation, plasma creatine 8.5 mg/dL, blood |
| | | | urea 142 mg/dL, bilateral optic nerve colobomas, and hypoplastic |
| | | | optic nerve in left eye. She had also a rarefied retina. Visual acuity |
| | | | was 20/200 in OD and CF in OS, history of febrile seizures with |
| | | | cognitive impairment in childhood. |
| III-16 | 4 | M | Vesicoureteral reflux at birth, bilateral optic nerve pits, visual |
| | | | acuity 20/20 in both eyes. |
| III-17 | 6 | M | Abdominal US showed bilateral small kidneys, bilateral cortical |
| | | | hyperechogenicity, poor corticomedullary differentiation, was |
| | | | submitted to renal transplantation in the beginning of 1998 (age 7) |
| | | | and is doing very well, serum creatinine 5.6 mg/dL, blood urea |
| | | | 124 mg/dL, bilateral optic nerve colobomas, visual acuity was |
| | | | 20/60 OD and 20/200 OS. |
| x2003 | 13 | M | Small kidneys with multiple small cysts on CT and MRI, |
| | | | progressive renal failure, bilateral cryptorchidism, bilateral optic |
| | | | nerve coloboma with retinal detachment |

As above, each exon of the PAX2 gene was amplified by PCR to identify a PAX2 mutation. A variant SSCP pattern was observed for exon 2, and sequencing of this PCR product revealed a guanosine nucleotide insertion at position 619 (FIG. 2D). The mutant and normal PAX2 sequence in exon 2 of the Japanese patient are shown in FIG. 2D. The mutation (arrowhead) is a G insertion resulting in an additional G (underlined) in a homonucleotide tract of 7 G's.

A list of the renal abnormalities identified in all 29 patients with PAX2 mutations, including the patients in this study, is given in Table 2.

TABLE 2
Renal abnormalities in 29 patients with PAX2 mutations

| | | | small | solitary | renal failure, proteinuria, | | renal |
|---|---|---|---|---|---|---|---|
| Patient | reference | age | kidney(s) | kidney | or abnormal histology | VUR | transplant |
| 1 | 5 | 15 | + | − | + | + | + |
| 2 | 5 | 10 | + | − | + | + | − |
| 3 | 5 | 6 | + | − | + | + | + |
| 4 | 5 | 35 | − | − | + | − | − |
| 430 | 7 | 5 | − | + | + | − | + |
| 431 | 7 | 12 | − | − | + | − | − |
| TRN | 8 | 5 | − | − | + | − | − |
| 579 | 9 | 11 | + | − | + | − | − |
| 656 | 9 | 48 | + | − | + | − | + |
| 657 | 9 | 25 | + | − | + | − | + |
| 2646 | 9 | 20 | + | − | + | − | + |
| III-8 | 10 | 70 | − | − | + | + | − |
| IV-2 | 11 | 52 | + | − | + | − | + |
| IV-3 | 11 | 46 | + | − | + | − | + |
| IV-6 | 11 | 40 | + | − | + | − | + |
| IV-7 | 11 | 35 | + | − | + | − | − |
| V-2 | 11 | 16 | + | − | + | − | − |
| F2 | 11 | 17 | + | − | + | + | − |
| 985 | 11 | 6 | − | − | + | + | − |
| I-1 | this study | 70 | − | − | + | − | − |
| II-5 | this study | 37 | + | − | + | − | − |
| II-7 | this study | 40 | − | − | − | − | − |
| II-9 | this study | 39 | − | − | + | − | − |
| III-9 | this study | 14 | − | − | − | − | − |
| III-11 | this study | 18 | − | − | − | − | − |
| III-13 | this study | 21 | + | − | + | − | − |
| III-16 | this study | 4 | − | − | + | + | − |
| III-17 | this study | 6 | + | − | + | − | + |
| X2003 | this study | 13 | + | − | + | − | − |

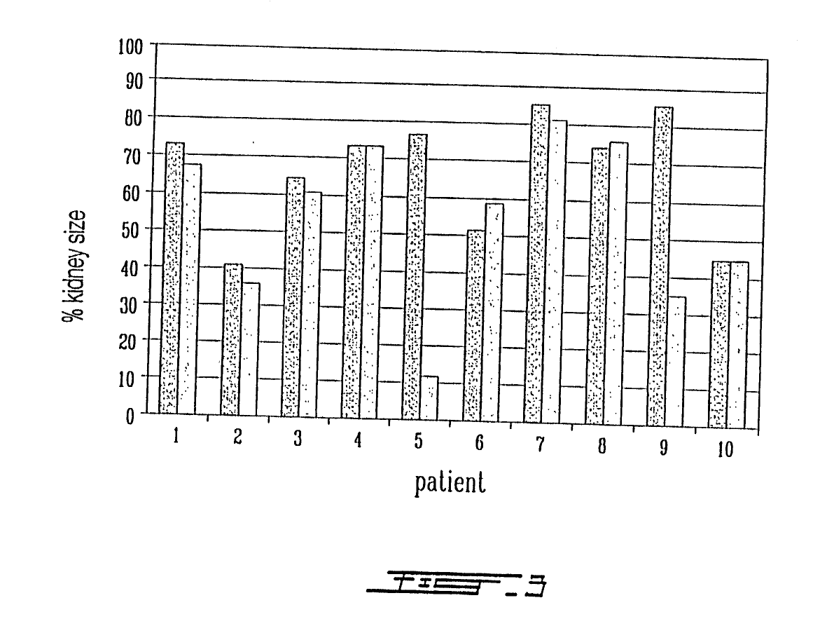

The most common renal abnormalities in the 29 patients were renal failure with histological abnormalities, proteinuria, and renal hypoplasia (small kidneys). Renal failure, histological abnormalities and proteinuria are features commonly seen in association with other disease processes, and are not specific for renal-coloboma syndrome. These abnormalities were probably secondarily acquired since birth. On the other hand, renal hypoplasia, although seen in association with a number of congenital conditions, is caused by failure of embryonic growth of the kidney, and would therefore be consistent with an abnormality arising primarily from the PAX2 mutation. Small kidneys were found in 17/29 patients (59%), and in 10 of these patients the sizes of the kidneys were measured by ultrasound and graphed as a percentage of the mean normal size of kidneys for individuals at that age (FIG. 3). In FIG. 3 kidney lengths are shown as a percentage of the mean normal kidney length for a person of the same age and sex; black bar, left kidney; grey bar, right kidney. The patients in order were 1, 3, TRN, 579, III-8, IV-2, IV-3, IV-6, IV-7, V-2 in Table 2.

Renal hypoplasia was observed even in the youngest patients in this study, consistent with it being a congenital abnormality associated with renal-coloboma syndrome.

Detailed analysis of the cause of the renal hypoplasia associated with renal-coloboma syndrome would have required characterization of fetal kidneys from these patients. This was not possible in human subjects, but a suitable mouse model of renal-coloboma syndrome (Pax2^{1Neu}) was available in which the mutation and syndrome were identical to those described in some humans (Koseki, C. et al. (1992) J. Cell Biol., 119, 1327-1333). We therefore undertook a detailed study of the kidney phenotype in heterozygous Pax2^{1Neu} mutant fetal mice.

### Renal Hypoplasia in Pax2^{1Neu} Heterozygous Mouse Embryos

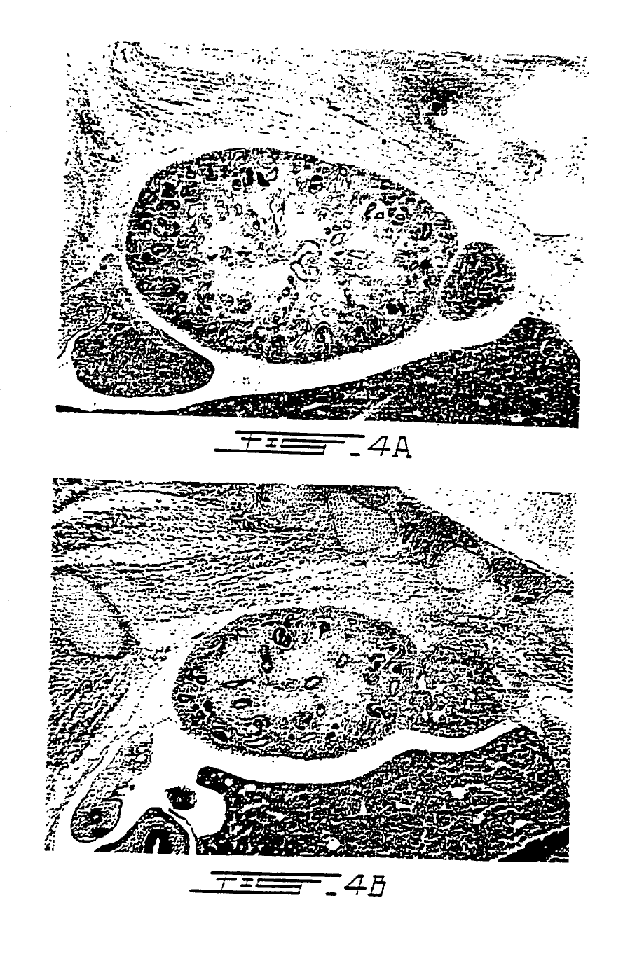

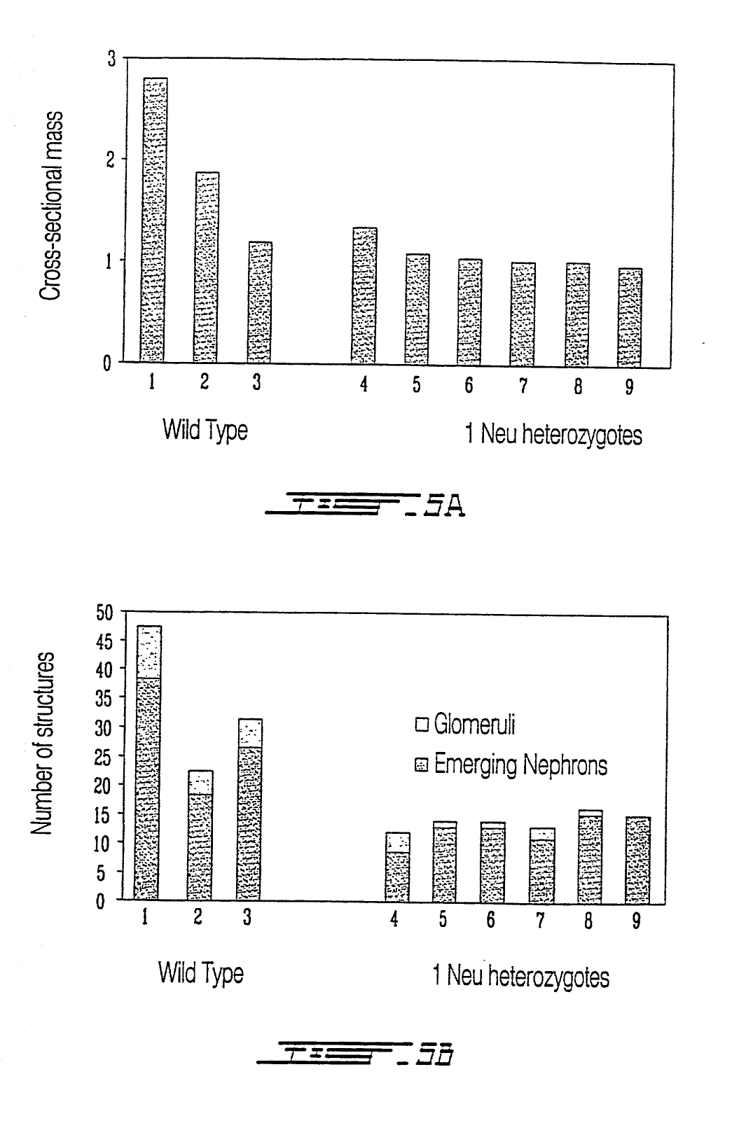

Homozygous Pax2^{1Neu} mice have renal agenesis, bilateral optic nerve colobomas, abnormalities of ear development and missing midbrain-hindbrain regions. These animals die within 24 hours of birth. However, heterozygous 1Neu mice, like their human counterparts, are viable and have no gross midbrain-hindbrain phenotype. Their main characteristics are abnormalities of optic nerve development and hypoplastic kidneys (u). Fetal mice carrying a heterozygous Pax2 mutation were obtained by crossing heterozygous Pax2^{1Neu} males with C57BL/6 females, and the fetuses were genotyped. Microscopic analysis of hematoxylin-eosin- stained sections from each embryo of three litters revealed that approximately 60% of the animals had hypoplastic kidneys with maximal cross-sectional surface area ranging from 30-75% of that of wild types. Representative litter is presented in FIGS. 4 and 5. Representative maximal cross-sections of E15 embryos from 1Neu +/− mutant and wild type littermates were stained with hematoxylin-eosin. Wild type kidneys show a complex nephrogenic zone and maturing medullary core (FIG. 4A). In contrast, 1Neu kidneys are clearly smaller with fewer nephrons and primitive medulla (FIG. 4B). Note the reduced number of mesenchymal condensates and ureteric bud branches in the nephrogenic zone of mutant kidney; mature glomeruli are absent on this representative section. Magnification×50. Maximal cross-sectional area of wild type and 1Neu heterozygous mutant kidneys were measured in a representative litter (arbitrary units; FIG. 5A). Mutant kidneys are smaller (60% of the average wild type cross-sectional area) but overlap with the smallest wild type kidneys. Numbers of mature glomeruli and early epithelial structures were measured in maximal cross-sections of kidneys from a representative litter FIG. 5B). Mutant kidneys have fewer mature glomeruli (22% of wild type) and early nephron structures (47% of wild type).

The largest heterozygous mutant kidneys overlapped in size with the smallest kidneys from wild type littermates. Unilateral renal agenesis was encountered in approximately 1% and cystic abnormalities were additionally observed in approximately 1% of animals.

The nephrogenic zones of fetal (E15-E16) 1Neu kidneys were thin and contained fewer nephrons in comparison to those of normal littermates. The number of early epithelial structures derived from induced metanephric mesenchyme (vesicles, comma- and S-shaped bodies) was reduced to 30-40% of normal (FIG. 5b). The number of mature glomeruli was even more sharply reduced (20% of the wild type). Interestingly, early tubular structures and glomeruli in Pax2 mutant kidneys appeared to be of normal size and morphology (FIG. 4).

### Rena Hypoplasia in Pax2^{1Neu} Mice is Associated with Enhanced Apoptosis of the Ureteric Epithelium

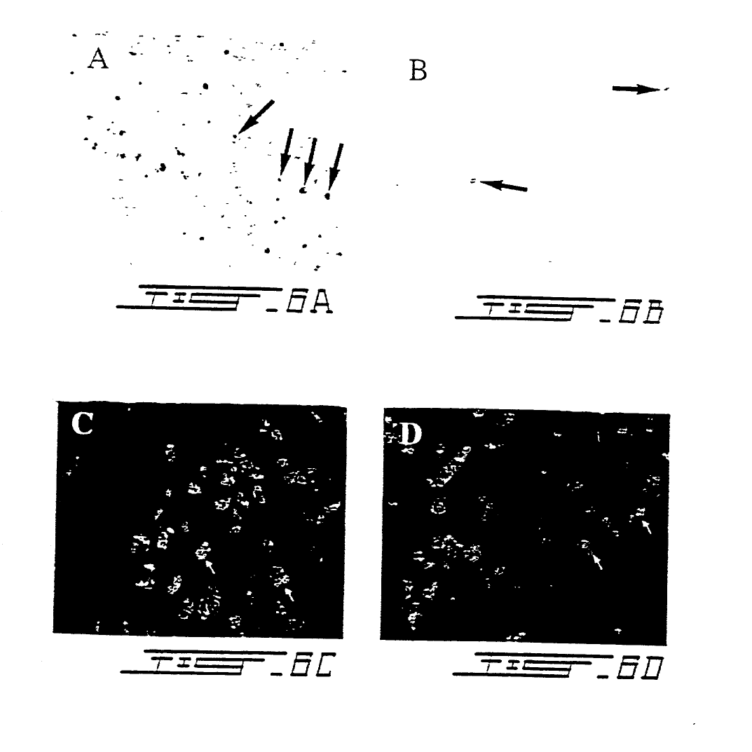

To gain insight into the mechanism of renal hypoplasia in Pax2 mutant mice, we examined the patterns of apoptotic cell death and cell proliferation in mutant and wild type fetal kidneys. TUNEL staining was used to investigate whether the Pax2 mutation in heterozygous Pax2^{1Neu} mutant mice was associated with increased apoptosis. An increase in TUNEL-positive staining was observed in the kidneys of embryonic day 15-16 (E15-16) Pax2^{1Neu}/+ mutant mice compared with in homozygous wild-type littermates (FIGS. 6A, 6B). Demonstration of apoptosis (arrowheads) in the collecting duct epithelia of kidneys from embryonic day 15 (E15) Pax2^{1Neu}/+ mutant mice (FIG. 6A), as compared with wild type E15 mice (arrows) (FIG. 6B) by TUNEL (TdT-mediated dUTP nick end labeling) staining (magnification 100×). Analysis of cell proliferation using BrdU incorporation (arrows) in the cortical region of kidneys from E15 Pax2^{1Neu}/+ mutant (FIG. 6C) and E15 wild type mice (FIG. 6D). The overall level of BrdU-labeling in mutant kidneys was similar to that in wild type kidneys (magnification 250×).

The rate of cell proliferation, as analyzed by BrdU uptake into DNA in the developing kidneys of mutant Pax2^{1Neu} /+ , was equivalent to that of wildtype mice from embryonic day E15 through postnatal day 6 (FIGS. 6C, 6D). Similarly, PCNA staining was unchanged in E15 1Neu embryos compared with their wild type littermates. These results suggest that heterozygosity for the 1Neu mutation is associated with an increase in the rate of apoptotic cell death but not with a difference in the pattern of fetal kidney cell proliferation.

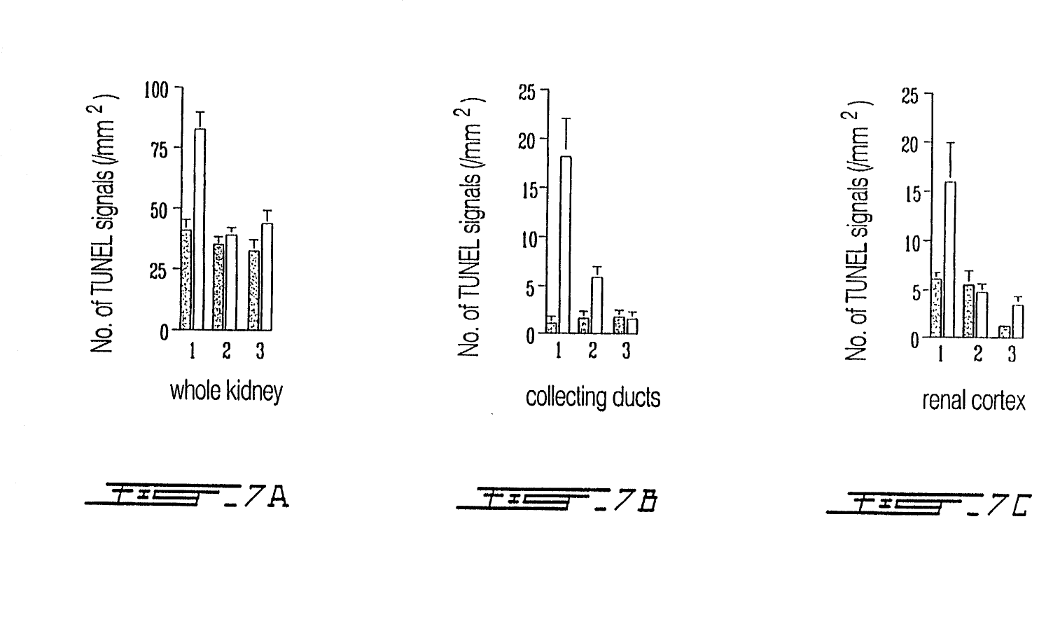

Apoptotic cell death in the mutant kidneys was examined in greater detail to identify the stages of development in which apoptosis was increased and the cell types involved. Although there were consistently more TUNEL-positive cells (p<0.001) in the total kidneys of E15-E16 Pax2^{1Neu}/+ mutant kidneys than in homozygous wild type littermates, this difference was less noticeable at later stages, and was confined to the collecting ducts (see below). In total kidneys of day E18, newborns (1 day-old), and in 5-6 day-old pups the number of TUNEL-positive cells were not significantly different in mutant and wild-type kidneys (FIG. 7A). The graphs show the number of TUNEL-stained cells in Pax2^{1Neu}/+ mutant (white bars) and wildtype mice (black bars) for (FIG. 7A) over the whole kidney, (FIG. 7B) specifically in collecting ducts, or (FIG. 7C) specifically in renal cortex. The X-axis shows mice analyzed at 3 different timepoints; E15-E16 (n=13 mutant and 9 wild type kidneys); E18-1 day old (d.o.) (n=13 mutant and 9 wild type kidneys); and 5-6 d.o (n=9 mutant and 11 wild type kidneys).

By counting apoptotic cells in specific renal structures of Pax2^{1Neu}/+ mutant kidneys a significant increase (p=0.0013) in TUNEL-positive staining was identified in the collecting ducts and renal pelvis in fetal (E15-E16) Pax2^{1Neu} mutants (FIG. 7b). This difference was still detectable in E18, but not in 5-6 day-old mice. TUNEL staining was also increased in the renal cortex in E15-E16 Pax2^{1Neu}/+ mutant fetal kidneys (FIG. 7C), which mostly corresponded to ureteric buds. Increased apoptosis was not observed in glomeruli, or proximal or distal tubules at either E15-E16 or later in development. The rate of apoptosis in fetal 1Neu kidneys was not determined prior to E15 because successfully mated females did not have vaginal plugs, and excessive numbers of randomly pregnant female mice would have been required to obtain the Figures for apoptotic rates.

### Pax2^{1Neu} Mutant Kidneys Contain Transcripts of both PAX2 Alleles and Express a Reduced Level of PAX2 Protein

Recent evidence by others suggests that another PAX gene, PAW5, is normally expressed stochastically from only one allele in developing lymphocytes. This observation raises the question as to whether PAX2 expression in developing kidney is also monoallelic. Accordingly, heterozygous mutant kidney would be comprised of a mixture of normal and null mutant cells. Apoptotic cell death might occur in those cells expressing the mutant allele, and viable collecting duct cells could consist of those randomly expressing normal amounts of PAX2 protein from the wild type allele. Alternatively, if PAX2 is expressed from both INeu alleles, then nephrons of heterozygous Pax2^{1Neu} kidneys should express PAX2 protein with perhaps a slight reduction in intensity.

To test the hypothesis that renal hypoplasia and the increased rate of apoptosis in 1Neu mutant fetal kidneys was associated with monoallelic Pax2 transcription we analyzed allelic patterns of Pax2 expression. The insertion mutation was used to discriminate the mutant and wild type alleles (FIG. 8A) in mRNA from heterozygous 1Neu mutant mouse kidneys following RT-PCR. Sequences depicting the G-insertion mutation in exon 2 of the Pax2^{1Neu} allele, the wild type Pax2 allele, and the locations of the primers (1F, 2F, 2R, 3R) used for PCR and RT-PCR. Reverse transcribed RNA was amplified using primers 1F and 3R (DNA amplification was precluded by introns 1 and 2). Primers 2F and 2R were used to amplify genomic DNA.

We reasoned that if Pax2 transcription were monoallelic in the developing kidney, cells randomly expressing the mutant allele might have a selective disadvantage and represent a diminishing portion of the kidney as development progressed. However, we found that levels of mutant and wild type Pax2 mRNA (alleles) were equivalent at all stages of development between fetal (E 12) and postnatal (day +1) kidney (FIG. 8B), suggesting that there was no selection for the favored expression of one allele in mutant kidneys. 6% denaturing PAGE gel showing mutant and wildtype Pax2 alleles in DNA and RNA (arrows). Lanes 1, 2, heterozygous 1Neu genomic DNA (lane 1) and wild type genomic DNA (lane 2); Lanes 3-9, RT-PCR of wild type (lane 3) and heterozygous mutant 1Neu fetal kidney mRNA (lanes 4-9). E12 fetal kidneys were in lanes 3, 4; E14 fetal kidneys were in lanes 8, 9; E18 fetal kidneys were in lanes 6, 7; postnatal (day +1) kidney was in lane 5.

In contrast to the above reasoning, cells monoallelically transcribing the mutant Pax2 allele might successfully proliferate, but these cells would not express the normal PAX2 protein. If there were large numbers of cells in the ureteric buds in the mutant kidneys not expressing immuno-reactive Pax2 protein, then this would be consistent with the notion that they express Pax2 mono-allelically from the mutant allele. We would be able to detect the resultant mosaicism (i.e. cells stamina positively and linegatively for PAX2 protein) by performing immunohistochemistry with a polyclonal anti-murine Pax2 antibody that reacts with C-terminal epitopes present in wild-type Pax2 protein, but which are absent in the truncated mutant Pax2 protein. Immunohistochemical staining for Pax2 protein was observed in all identifiable nuclei of ureteric bud and collecting duct cells of heterozygous 1Neu fetal kidneys (FIG. 8C), but the intensity of Pax2 staining was uniformly less than that in fetal kidneys of wild-type littermates analyzed simultaneously. Immunohistochemistry of E15 heterozygous mutant 1Neu mouse fetal kidney, and FIGS. 8C D, E15 wild type mouse fetal kidney using anti-Pax2 primary antibody. Nuclei stained brown in the sections correspond to cells of the collecting duct and ureteric bud expressing the Pax2 protein.

By comparison, strong staining for Pax2 was observed in all portions of wild type ureteric buds and collecting ducts (FIG. 8D). This pattern of staining was observed in each of three wild-type and three mutant embryos analyzed simultaneously. These results suggest that all the cells of the ureteric buds expressed the wild-type Pax2 allele. To examine PAX2 allelic expression patterns directly in clonal cell populations we analyzed transcription in Wilms tumors and in cloned renal carcinoma cells using a polymorphism in human PAX2 (FIG. 8E). Depiction of the C/A polymorphism in exon 8 used to distinguish PAX2 aileles during amplification of human PAX2 exon 8 mRNA from Wilms tumors and renal carcinoma cells, and the locations of the primers (P1, P2, P3, P4) used for RT-PCR and DNA PCR. Reverse transcribed RNA was amplified using primers P1 and P4 (DNA amplification was precluded by introns 7 and 8), and RT-PCR products were analyzed by SSCP. Genomic DNA for genotyping was amplified using primers P2 and P3.

RT-PCR-SSCP was used to discriminate between the two PAX2 alleles (FIG. 8f, lanes 1-3). Biallelic transcription of PAX2 in fetal kidney (FIG. 8F, lane 7) was not unexpected since monoallelic transcription of PAX2 in one cell (if present) would be statistically matched by monoallelic transcription of the opposite allele in another cell. RT-PCR-SSCP gels showing biallelic PAX2 expression in human fetal kidney and tumors. SSCP revealed four conformations for the two alleles (upper and lower arrows). Lanes 1-3, fetal kidneys homozygous for one allele (lane 1), heterozygous (lane 2), or homozygous for the other allele (lane 3). Lanes 4-6, heterozygous Wilms tumors. Lane 7, heterozygous human fetal kidney (12 weeks gestation). Lanes 8, 9, renal carcinoma cell line (A704) before cell cloning (lane 8), and after cell cloning (lane 9). Primer sequences; exon 8; P1, 5′-AGC TTT GGA TCG GGT CTT TGA-3′; P2, 5′-CCT TTC TCT GTG CGT GCA TCA ATA GA-3′; P3, 5′-GGC ACC CTC CAC TGA ACG CAG-3′; P4, 5′-CAG GGT GGA GGT GGG GTA G-3′.

However, Wilms tumors are predominantly comprised of clonal blastemal and epithelial tissues, derived originally from embryonic kidney. Biallelic PAX2 transcription was observed in mRNA isolated from a series of 3 Wilms tumor tissues (FIG. 8f, lanes 4-6). In addition, biallelic (albeit slightly unbalanced) PAX2 transcription was observed in clonally expanded renal carcinoma cells (FIG. 8F, lanes 8, 9). The small imbalance of PAX2 allelic expression observed in the cells may be due to alterations in the number of copies of each PAX2 allele in the cells, and was an indication that the RT-PCR-SSCP technique was sensitive enough for this purpose.

### Discussion

In accordance with the present invention, we have further characterized the renal phenotype associated with renal-coloboma syndrome and have identified elevated levels of apoptosis in fetal kidneys of individuals carrying a PAX-2 mutation. End-stage renal failure is commonly associated with this syndrome, and one goal of this research was to determine how PAX2 mutations could cause renal failure. This was addressed firstly by characterizing the renal phenotype in renal-coloboma syndrome patients. Renal hypoplasia was the most common congenital abnormality in a series of 29 patients, including 10 new patients reported here.

Of the ten new patients identified with renal-coloboma syndrome, nine were from a large 3-generation Brazilian family transmitting a novel C→T substitution at position 1289 of PAX2, resulting in a change from an arginine codon in exon 7 to a stop codon. The mutation identified in the Brazilian family is the most 3′ mutation so far identified in PAX2, and is the first report of a PAX2 mutation which leads directly to the introduction of a stop codon. If expressed, the protein resulting from this mutant allele is predicted to be truncated midway through the partial homeodomair., and would result in loss of the entire transactivation domain. PAX2 protein lacking the transactivation domain is unlikely to be fully functional, as studies have shown that this region in the closely related PAX5 protein is required for transcriptional activation of target genes. In the tenth patient, a Japanese boy, we identified a G619 insertion mutation of PAX2 exon 2. The G619 insertion mutation has previously been documented in other patients (Favor, J. et al. (1996) Proc. Natl. Acad. Sci. USA, 93, 1380-1387; Gavrielli, Y. et al. (1992) J. Cell Biol., 119, 493-501). This patient also presented with bilateral cryptorchidism, which has not previously been described in renal-coloboma syndrome and may be just a chance association. However, Pax2 is expressed in the developing urogenital ridge, vas deferens, and epidydimis (Dressler, G. R. et al. (1990) Development, 109, 787-795), and these structures are important in the maturation of the genital tract. It is possible that PAX2 mutations cause less obvious abnormalities of the reproductive system in other patients with renal-coloboma syndrome.

Four of the ten new patients in this report had obvious renal hypoplasia; three others had clinical evidence of renal insufficiency. When we reviewed clinical data on the previously reported 19 cases of proven human PAX2 mutations, we noted 13/19 with obvious renal hypoplasia and all had evidence of renal insufficiency or dysfunction. In at least two of the three cases where renal biopsy had been performed in childhood, there was striking atrophy of the proximal and distal tubules, but nephron number could not be assessed. Renal hypoplasia was pronounced even in the youngest patients in this study (patients 2 and 3), who were 5 and 6 years old, suggesting that this is a congenital anomaly.

Further studies to determine how PAX2 mutations cause renal hypoplasia in human subjects were impractical because it was not possible to obtain fetal kidneys from patients with renal-coloboma syndrome. However, Pax2^{1Neu} mutant mice (Koseki, C. et al. (1992) J. Cell Biol., 119, 1327-1333) harbor a mutation that is identical to a mutation in approximately one third of families with renal-coloboma syndrome. These mice transmit a single base pair insertion amid a string of seven cuanidine residues (positions 613-619) in the second exon of Pax2, which produces a frameshift and presumably a null allele (Koseki, C. et al. (1992) J. Cell Biol., 119, 1327-1333). Pax2^{1Neu} mice have optic nerve and kidney abnormalities, similar to humans with renal-coloboma syndrome. Although the homozygous mutants die within 24 hours of birth and are phenotypically similar to “knockout” mice generated by targeted homologous recombination, heterozygotes survive and reach adulthood. The heterozygous mutant mice therefore afford a useful animal model in which to analyze fetal kidney defects associated with Pax2 mutations.

During normal kidney development Pax2 is first detected along the nephrogenic cord at the sites from which ureteric buds will emerge (Dressler, G. R. et al. (1990) Development, 109, 787-795). Intense Pax2 expression persists in cells of the ureteric buds as they invade the metanephric blastema to each side and begin to undergo dichotomous branching (Dressier, G. R. et al. (1990) Development, 109, 787-795, Eccles, M. R. et al. (1992) Cell Growth & Diff., 3, 279-289). Inspection of fetal Pax2^{1Neu}/+ mouse kidneys demonstrated renal hypoplasia at an early (E15) stage of development. This is apparently due to a decrease in the rate of new nephron induction since the total number of early epithelial structures (at the tips of ureteric buds) and glomeruli (representing more advanced nephrons) is strikingly reduced in mutant kidneys. The nephrons that are formed, though reduced in number, appear to have normal morphology. It can be inferred, therefore, that arborization of the ureteric bud is less complex in patients with heterozygous PAX2 mutations than in normal individuals. It should be pointed out that even a modest reduction in the efficiency of ureteric bud branching would reduce final kidney size substantially, since the dichotomous branching process is repeated many times during renal development.

The downstream gene targets of PAX2 are largely unknown, but appear to influence growth and branching morphogenesis of the ureteric bud. For that reason we examined the pattern of cell proliferation and apoptotic cell death in embryonic kidneys of heterozygous Pax2^{1Neu} mutant mice. Cell division, as assessed by PCNA immunohistochemistry and BrdU uptake, was normal. However, apoptosis in the medullary segments of the collecting duct (as assessed by TUNEL assay) was strikingly increased at the time when Pax2 is maximally expressed in kidney development, and in cells which normally express Pax2 (Dressler, G. R. et al. (1990) Development, 109, 787-795). Indeed, the amount and localization of the apoptosis correlated temporally and spatially with the known expression patterns of PAX2 (Dressler, G. R. et al. (1990) Development, 109, 787-795, Eccles, M. R. et al. (1992) Cell Growth & Diff., 3, 279-289), except that apoptosis was not observed in mesenchyme-derived ezithelial structures, whereas PAX2 is expressed in the differentiating mesenchyme and early mesenchyme-derived structures. E15 mutant mice exhibited the greatest levels of apoptosis, and at later stages when overall PAX2 expression levels have declined (Dressler, G. R. et al. (1990) Development, 109, 787-795), differences in the rate of apoptosis in collecting ducts was less marked between heterozygous Pax2^{1Neu}/+ mutant and wild type kidneys. Apoptosis is known to occur as part of normal kidney development, but the rate of apoptosis that we observed in E15 collecting ducts was approximately 9-fold higher than in wild type offspring at the same age. Since apoptotic cells are rapidly cleared by phagocytosis, we can not ascertain whether cell death is extensive enough to compromise arborization of the ureteric bud. Conceivably, our observation of increased apoptosis may reflect reduced signaling by trophic factors influencing branching morphogenesis as well as cell survival.

It is unclear why the apoptosis in the heterozygous mutant kidneys was predominantly confined to Wollfian duct-derived structures such as ureteric buds and collecting ducts, even though Pax2 is also expressed in mesenchymally-derived structures such as comma- and S-shaped bodies. One possible explanation arises from the observation that Pax8 is co-expressed with Pax2 in the differentiating mesenchyme-derived structures, but not in ureteric buds or collecting ducts. It is possible that, as has been described for other Pax genes, expression of Pax8 was able to compensate for heterozygous Pax2 mutations in the mesenchymally-derived structures of kidneys from Pax2 mutant mice, and that this ultimately rescued these cells from apoptosis.

Our data do not determine whether PAX2 directly regulates genes in apoptotic pathways during kidney development. However, PAX2, PAX5 and PAX8 have all been reported to inhibit p53 transcription, which can promote apoptosis. Furthermore, transgenic mice overexpressing wild type p53 have altered differentiation of the ureteric bud, and have small kidneys. In Pax5 knockout mice the B cells fail to differentiate and undergo apoptotic cell death at an early stage. Similarly, large-scale apoptosis of photoreceptor precursors occurs in the eye discs of Drosophila with the eyeless mutation (ey²) (homologous to the mammalian Pax6 gene). These authors also found no difference in BrdU uptake studies in wild type and mutant eye discs. As in our studies, this suggests that the mutation affects cell survival rather than proliferation. Pax3 mutant mice were shown to have enhanced apoptosis in the somatic mesoderm during embryonic development, and treatment of rhabdomyosarcoma cells lines with PAX3 antisense oligonucleotides has been shown to result in apoptosis. Unlike the above studies reporting apoptosis in homozygous Pax mutant animals, our studies were carried out on heterozygous mutant animals.

Others speculated that Pax2+/− mice might develop renal hypoplasia due to a decreased rate of cell proliferation in renal calyces and upper ureters. However, our results suggest that the cause of the small kidneys in renal-coloboma syndrome is due to decreased cell survival in these structures rather than decreased proliferation. The fact that the collecting system is derived from the Wolffian duct, and that we observed apoptosis at the earliest time-point examined (E15), suggests that apoptosis could have occurred in the Wolffian duct while the ureteric bud was sprouting from it. This notion would be consistent with, and may help to explain the observed degeneration and lack of caudal extension of the Wolffian duct in Pax2 null mutant mice. In Pax2 mutants the lack of development of, or dysplasia of the epidydimis, vas deferens, seminal vesicles and ureters, each of which are derived from the Wolffian duct, could also be caused by enhanced apoptosis in the Wolffian duct and its derivatives.

The effects of PAX2 haploinsufficiency on the renal phenotype are similar to the developmental effects of haploinsufficiency for other PAX genes. PAX3 mutations cause an autosomal dominant defect in epidermal pigmentation, PAX6 mutations cause autosomal dominant aniridia, and PAX8 mutations cause autosomal dominant hypothyroidism. In each case, inactivation of one allele is sufficient to interfere with normal organ development. With regard to the mechanism of haploinsufficiency, others have hypothesized that monoallelic Pax gene expression may be related to the semi-dominant effects of Pax gene mutations. We analyzed the allelic expression pattern of Pax2 in 1Neu mutant kidneys both by RT-PCR, and by immunostaining. In Pax2 mutant kidneys, immunostaining of Pax2 was detected in virtually every cell in the ureteric buds. Taken together with our RT-PCR data, these observations suggest that Pax2 is biallelically transcribed in wild-type fetal kidney, and that ureter and collecting duct abnormalities produced by Pax2 haploinsufficiency probably result from sensitivity to reduced gene dosage in each cell. We can not rule out the possibility that Pax2 is monallelically expressed at embryonic stages earlier than E15, or in stem cells which were not amenable to study by immunohistochemistry in E15 kidney.

In summary, the murine Pax2^{1Neu} mutation adds much to our understanding of the human disease. We have shown that renal hypoplasia is the most common congenital renal anomaly in humans and mice with PAX2 mutations, and that in mice this phenotype is associated with significantly enhanced apoptosis. Furthermore, the Pax2 gene was found to be biallelically expressed in mutant mice, therefore it seems unlikely that the enhanced apoptosis was due to random stochastic inactivation of Pax2 expression in cells mono-allelically expressing Pax2. We conclude from this work that haploinsufficiency of Pax2 leads to small kidneys and renal failure because reduced Pax2 dosage compromises the survival of ureteric bud-derived epithelial cells, and that this is associated with reduced branching of the ureteric bud at an early stage of development. Further studies are required to determine if PAX2 is a primary determinant of cell survival, or whether this effect is the consequence of a default pathway of apoptosis in cells in which development is disrupted.

While the invention has been described in connection with specific embodiments thereof, it will be understood that it is capable of further modifications and this application is intended, to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method of modulating resistance to apoptosis in a patient, comprising administering to said patient a therapeutically effective amount of enhancing agent which selectively inhibits and/or prevents the function of PAW-2.

2. The method of claim 1, wherein said enhancing agent inhibits the expression of PAS-2 gene.

3. The method of claim 1, wherein said enhancing agent is an antisense oligonucleotide of PAX-2 gene.

4. The method of claim 1, wherein said enhancing agent is an antisense oligonucleotide of PAX-2 RNA primary transcript and/or RNA messenger.

5. The method of claim 1, wherein said enhancing agent inhibits the activity of PAX-2 protein and/or binding of PAX-2 protein to PAX-2 binding site.

6. The method of claim 1, wherein said enhancing agent is selected from the group of ribozyme, decoy oligonucleotide.

7. The method according to claim 5, wherein said enhancing agent is selected from the group consisting of antibody, abzyme, inactive mutant, inactive homologue, inactive analogue, or inactive fragment of PAX-2, and derivatives thereof.

8. The method according to claim 1, wherein said modulation is modulating resistance to apoptosis of cancer cells or cystic kidney cells.

9. The method according to claim 8, wherein said cancer cells are selected from the group consisting of prostate cancer, ovarian cancer, bladder cancer, and kidney cancer and/or cystic kidney diseases in which PAX-2 is expressed at higher level than in a healthy patient.

10. A method of rescuing cells from apoptosis in a patient comprising the step of administering to said patient a therapeutically effective amount of an enhancing agent which selectively stimulates the function of PAX-2.

11. The method of claim 10, wherein said enhancing agent stimulates the expression of PAX-2 gene.

12. The method according to claim 11, wherein said enhancing agent activates the expression of the PAX-2 gene by activating the PAX-2 gene promoter and/or PAX-2 gene transcription regulatory elements.

13. A method to enhance resistance of normal tissues to apoptotic cell death induced by chemotherapy or radiation therapy in a patient comprising the step of administering to said patient a therapeutically effective amount of an enhancing agent which selectively stimulates the function of PAX-2.

14. The method of claim 13, wherein said enhancing agent activates the expression of PAX-2 gene.

15. The method according to claim 13, wherein said enhancing agent activates the expression of the PAX-2 gene by activating the PAX-2 gene promoter and/or PAX-2 gene transcription regulatory elements.

16. A genetic construct comprising a nucleic acid encoding a molecule capable of preventing or stimulating the function of PAX-2.

17. The genetic construct according to claim 16, wherein said enhancing agent is selected from the group consisting of antisense oligonucleotide, ribozyme, antibody, abzyme, mutant, homologue, analogue, or fragment of PAX-2, PAW-2 transcription regulatory factor, and derivatives thereof.

18. Use of an enhancing agent for the preparation of a medicament for modulating resistance to apoptosis in a subject, wherein said enhancing agent selectively inhibits and/or prevents the function of PAW-2 gene.

19. The use of claim 18, wherein said enhancing agent inhibits the expression of PAX-2 gene.

20. The use of claim 18, wherein said enhancing agent is an antisense oligonucleotide of PAX-2 gene.

21. The use of claim 18, wherein said enhancing agent is an antisense oligonucleotide of PAX-2 RNA primary transcript and/or RNA messenger.

22. The use of claim 18, wherein said enhancing agent inhibits the activity of PAX-2 protein and/or binding of PAX-2 protein to PAX-2 binding site.

23. The use of claim 18, wherein said enhancing agent is selected from the group of ribozyme, decoy oligonucleotide.

24. The use according to claim 22, wherein said enhancing agent is selected from the group consisting of antibody, abzyme, inactive mutant, inactive homologue, inactive analogue, or inactive fragment of PAX-2, and derivatives thereof.

25. The use according to claim 18, wherein said modulation is modulating resistance to apoptosis of cancer cells or cystic kidney cells.

26. The use according to claim 25, wherein said cancer cells are selected from the group consisting of prostate cancer, ovarian cancer, bladder cancer, and kidney cancer and/or cystic kidney diseases in which PAX-2 is expressed at higher level than in a healthy subject.

27. Use of an enhancing agent for the preparation of a medicament for the rescuing of cells from apoptosis in a subject, wherein said enhancing agent selectively stimulates the function of PAX-2 gene.

28. The use of claim 27, wherein said enhancing agent stimulates the expression of PAX-2 gene.

29. The use according to claim 28, wherein said enhancing agent activates the expression of the PAX-2 gene by activating the PAX-2 gene promoter and/or PAX-2 gene transcription regulatory elements.

30. Use of an enhancing agent for the preparation of a medicament for enhancing resistance of normal tissues to apoptotic cell death induced by chemotherapy or radiation therapy in a subject, wherein said enhancing agent selectively stimulates the function of PAX-2 gene.

31. The use of claim 30, wherein said enhancing agent activates the expression of PAX-2 gene.

32. The use according to claim 30, wherein said enhancing agent activates the expression of the PAX-2 gene by activating the PAX-2 gene promoter and/or PAX-2 gene transcription regulatory elements.
